(19) United States
(12) Patent Application Publication
(10) Pub. No.: US 2005/0019909 A1
(43) Pub. Date: Jan. 27, 2005

(54) Modified retinal pigment epithelial cells for cell transplantation

(75) Inventor: Thyagarajan Subramanian, Westlake, OH (US)

Correspondence Address: TAROLLI, SUNDHEIM, COVELL & TUMMINO L.L.P., SUITE 1111, 526 SUPERIOR AVENUE, CLEVELAND, OH, 44114-1400, US

(73) Assignee: The Cleveland Clinic Foundation

(21) Appl. No.: 10/461,941

(22) Filed: Jun. 13, 2003

Related U.S. Application Data
(60) Provisional application No. 60/388,333, filed on Jun. 13, 2002.

(51) Int. Cl. A61K 48/00; C12N 5/08

(52) U.S. Cl. 435/368; 424/93.21

(57) ABSTRACT

A transplantable composition comprises retinal pigment epithelium cells modified to induce expression and/or secretion of a therapeutic molecule, that can be used to prevent and/or treat disease.

The present application claims priority to U.S. Provisional Application No. 60/388,333 filed Jun. 13, 2002, which is incorporated herein by reference in its entirety.

The work described in this application was supported, at least in part, by Grant No. NINDS R01. The United States government has certain rights in this invention.

### FIELD OF THE INVENTION

The present invention relates to retinal pigment epthelial (RPE) cells and particularly to modified RPE (mRPE) cells and to their use in the prevention and treatment of neurological disorders.

### BACKGROUND OF THE INVENTION

Cell transplantation is an experimental therapy for Parkinson's disease (PD) and other movement disorders. Several open label research trials have shown clinically meaningful improvement in parkinsonian signs and symptoms after striatal transplantation of allogenic fetal ventral mesencephalic (FVM) tissue. However, ethical concerns, variability in surgical techniques and reports of unusual late complications in a few patients in a recently completed clinical trial have limited the use of allogenic FVM tissue to a few research centers. Research into alternatives cell sources like porcine FVM and allogenic retinal pigment epithelial cells have shown promising results in preclinical trials and are currently being tested in clinical trials. Novel strategies to improve cell survival and to avoid immune rejection of transplants show promising results in preclinical trials. Deep brain stimulation (electrical stimulation of the brain) is also being investigated as a method of treating patients with advanced Parkinson's disease and in other neurological disorders.

### SUMMARY OF THE INVENTION

The present invention relates to retinal pigment epithelial cells that are modified to induce expression and/or secretion of therapeutic molecules, such as therapeutic proteins. Protein expression and/or secretion can modified using viral and/or non viral mediated gene transfer techniques to introduce nucleic acids (e.g., DNA) into human and animal RPE cells in vitro to facilitate expression of proteins (e.g., native and non-native) that are not normally expressed in RPE cells or are expressed in very low levels. Such increased expression of proteins enhances the ability of RPE cells to produce large quantities of therapeutic molecules (e.g., proteins, such as dopamine and/or growth factors like glial derived neurotrophic factor (GDNF)). This enhanced ability makes modified RPE (MRPE) cells a more desirable product for cell transplantation. MRPE can be used for cell transplantation therapy for neurological diseases, such as Parkinson's disease, Huntington's disease, stroke, spinal cord injury, post-polio syndrome amyotrophic lateral sclerosis, epilepsy and other human diseases, as well as other diseases.

### BRIEF DESCRIPTION OF THE DRAWINGS

Further features of the present invention will become apparent to those skilled in the art to which the present invention relates from reading the following description of the invention with reference to the accompanying drawings in which:

FIG. 1 is a graph showing the beneficial effects of RPE cells following transplantation to treat Parkinson's disease.

FIG. 2 is a photograph showing histological sections of a mouse brain following RPE transplantation.

FIG. 3 is a photograph showing histological sections of a mouse brain following RPE transplantation.

FIG. 4 is a schematic illustration showing of a possible mechanism of dopamine synthesis and release.

FIG. 5 is a schematic illustration showing the pathway of melanin synthesis.

FIG. 6 is a photograph comparing the immunochemistry of PC-12, FVM, and rat brain RPE cells for TH staining and DDC staining as well as VMAT-2 and DAT.

FIG. 7 are western blots comparing the expression of TH protein, AADC, and tyrosinase in PC-12 cells and RPE cells.

FIG. 8 is a western blot comparing the expression of VMAT in SKNMC cells and RPE cells.

FIG. 9 is a schematic illustrate of a MLV based retroviral vector plasmid

FIG. 10 is a photograph illustrating DDC immunohistochemisty. a. ARPE-19 cells. b. ARPE transfected with lentiDDC, c. PC-12 cells, d. 6-OHDA lesioned rat striatum

FIG. 11 is a picture illustrating GFP transfection after 1 week. a. nonviral method, b. MLV based retroviral method, c. lentiviral method

FIG. 12 is a photograph illustrating long-term GFP expression in ARPE-19 cells. Left. GFP positive cells after MLV based retroviral infection and puromycin selection, Right. Lentiviral infection.

FIG. 13 is a photograph illustrating confocal images of GFP positive cells (green), DDC positive cells (red) and cells co-expressing DDC and GFP (yellow) in ARPE-19 culture 1 week after infecting with lenti-GFP and 6 days after lenti-DDC infection.

### DESCRIPTION OF THE EMBODIMENTS

Unless otherwise defined, all technical terms used herein have the same meaning as commonly understood by one of ordinary skill in the art to which this invention belongs. Commonly understood definitions of molecular biology terms can be found in, for example, Rieger et al., Glossary of Genetics: Classical and Molecular, 5th edition, Springer-Verlag: New York, 1991; and Lewin, Genes V, Oxford University Press: New York, 1994.

Methods involving conventional molecular biology techniques are described herein. Such techniques are generally known in the art and are described in detail in methodology treatises, such as Molecular Cloning: A Laboratory Manual, 2nd ed., vol.1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Methods for chemical synthesis of nucleic acids are discussed, for example, in Beaucage and Carruthers, Tetra. Letts. 22:1859-1862, 1981, and Matteucci et al., J. Am. Chem. Soc. 103:3185,1981. Chemical synthesis of nucleic acids can be performed, for example, on commercial automated oligonucleotide synthesizers. Immunological methods (e.g., preparation of antigen-specific antibodies, immunoprecipitation, and immunoblotting) are described, e.g., in Current Protocols in Immunology, ed. Coligan et al., John Wiley & Sons, New York, 1991; and Methods of Immunological Analysis, ed. Masseyeff et al., John Wiley & Sons, New York, 1992. Conventional methods of gene transfer and gene therapy can also be adapted for use in the present invention. See, e.g., Gene Therapy: Principles and Applications, ed. T. Blackenstein, Springer Verlag, 1999; Gene Therapy Protocols (Methods in Molecular Medicine), ed. P. D. Robbins, Humana Press, 1997; and Retro-vectors for Human Gene Therapy, ed. C. P. Hodgson, Springer Verlag, 1996.

The present invention relates to retinal pigment epithelial cells that are modified to induce expression and/or secretion of therapeutic molecules, such as therapeutic proteins. Retinal pigment epithelial cells can modified in vitro facilitate expression of therapeutic molecules (e.g., native proteins and/or non-native proteins) that are not normally expressed in RPE cells or are expressed in very low levels. Such increased expression of proteins enhances the ability of RPE cells to produce large quantities of therapeutic molecules (e.g., proteins, such as dopamine and/or growth factors like glial derived neurotrophic factor (GDNF)). This enhanced ability makes modified RPE (MRPE) cells a more desirable product for cell transplantation.

In accordance with one aspect of the present invention, the RPE cells can be modified using viral and/or non viral mediated gene transfer techniques to introduce nucleic acids (e.g., DNA or genes) into human and animal RPE cells in vitro to facilitate expression of therapeutic molecules (e.g., therapeutic proteins)). One example of a gene transfer technique uses a vector including a nucleotide encoding for or inducing expression of the protein. A “vector” (sometimes referred to as gene delivery or gene transfer “vehicle”) refers to a macromolecule or complex of molecules that is capable of delivering the nucleic acid to the target cell, either in vitro or in vivo. The nucleic to be delivered may comprise a coding sequence of interest in gene therapy.

The vector can include other macromolecular complexes capable of mediating delivery of the nucleic acid to the RPE cell. The vector can also comprise other components or functionalities that further modulate gene delivery and/or gene expression, or that otherwise provide beneficial properties to the RPE cells. Such other components can include, for example, components that influence binding or targeting to; components that influence uptake of the vector nucleic acid by the cell; components that influence localization of the nucleic acid within the cell after uptake (such as agents mediating nuclear localization); and components that influence expression of the nucleic acid. Such components also might include markers, such as detectable and/or selectable markers (e.g., a reporter gene, such as GFP) that can be used to detect or select for cells that have taken up and are expressing the nucleic acid delivered by the vector. Such components can be provided as a natural feature of the vector (such as the use of certain viral vectors, which have components or functionalities mediating binding and uptake), or vectors can be modified to provide such functionalities.

Selectable markers can be positive, negative or bifunctional. Positive selectable markers allow selection for cells carrying the marker, whereas negative selectable markers allow cells carrying the marker to be selectively eliminated. A variety of such marker genes have been described, including bifunctional (i.e., positive/negative) markers (see, e.g., Lupton, S., WO 92/08796, published May 29, 1992; and Lupton, S., WO 94/28143, published Dec. 8, 1994). Such marker genes can provide an added measure of control that can be advantageous in gene therapy contexts. A large variety of such vectors are known in the art and are generally available.

Vectors for use in the present invention include viral vectors and non-viral vectors, such as lipid based vectors and other vectors that are capable of delivering the nucleic acid in accordance with the present invention to the RPE cells.

Presently preferred viral vectors can include lentil viral vectors. Lentiviral vectors are advantageous in that they are highly efficient at transducing human epithelial cells. Lentiviral vectors for use in the invention may be derived from human and non-human (including SIV) lentiviruses. Lentiviral vectors include nucleic acid sequences required for vector propagation as well as a promoter operably linked to the gene used to express the therapeutic protein. These former may include the viral LTRs, a primer binding site, a polypurine tract, att sites, and an encapsidation site.

A lentiviral vector may be packaged into any suitable lentiviral capsid. The substitution of one particle protein with another from a different virus is referred to as “pseudotyping”. The vector capsid may contain viral envelope proteins from other viruses, for example, murine leukemia virus (MLV), vesicular stomatitis virus (VSV), and rabies-G.

Other examples of viral vectors that can be used in accordance with the present invention include adenovirus (Ad) based vectors, adeno-associated virus (AAV) based vectors, herpes simplex virus (HSV)-based vectors, and retroviruses, such as C-type retroviruses. Alphavirus-based vectors, such as those made from semliki forest virus (SFV) and sindbis virus (SIN), might also be used in the invention. Use of alphaviruses is described in Lundstrom, K., Intervirology 43:247-257, 2000 and Perri et al., Journal of Virology 74:9802-9807, 2000. Alphavirus vectors typically are constructed in a format known as a replicon.

In many of the viral vectors compatible with the present invention, more than one promoter can be included in the vector to allow more than one heterologous gene to be expressed by the vector. Further, the vector can comprise a sequence which encodes a signal peptide or other moiety which facilitates the secretion of a therapeutic gene product (i.e., GDNF) from the RPE cells.

To combine advantageous properties of two viral vector systems, hybrid viral vectors may be used to deliver a nucleic acid to RPE cells. Standard techniques for the construction of hybrid vectors are well-known to those skilled in the art. Such techniques can be found, for example, in Sambrook, et al., In Molecular Cloning: A laboratory manual. Cold Spring Harbor, N.Y. or any number of laboratory manuals that discuss recombinant DNA technology. Double-stranded AAV genomes in adenoviral capsids containing a combination of AAV and adenoviral ITRs may be used to transduce cells. In another variation, an AAV vector may be placed into a “gutless”, “helper-dependent” or “high-capacity” adenoviral vector. Adenovirus/AAV hybrid vectors are discussed in Lieber et al., J. Virol. 73:9314-9324, 1999.

In addition to viral vectors, non-viral vectors may also be used to introduce a gene into the RPE cells. A review of non-viral vectors and non-viral vector based methods of gene delivery is provided in Nishikawa and Huang, Human Gene Ther. 12:861-870, 2001. One example of a non-viral vector according to the invention employs plasmid DNA to introduce a nucleic acid into a cell. Plasmid-based gene delivery methods are generally known in the art.

Synthetic gene transfer molecules can be designed to form multimolecular aggregates with plasmid DNA. Cationic amphiphiles, including lipopolyamines and cationic lipids, may be used to provide receptor-independent nucleic acid transfer into the RPE cells. In addition, preformed cationic liposomes or cationic lipids may be mixed with plasmid DNA to generate cell-transfecting complexes. Methods involving cationic lipid formulations are reviewed in Feigner et al., Ann. N.Y. Acad. Sci. 772:126-139,1995 and Lasic and Templeton, Adv. Drug Delivery Rev. 20:221-266,1996. For gene delivery, DNA may also be coupled to an amphipathic cationic peptide (Fominaya et al., J. Gene Med. 2:455-464, 2000).

Vectors that involve both viral and non-viral based components may be used according to the invention. For example, an Epstein Barr virus (EBV)-based plasmid for therapeutic gene delivery is described in Cui et al., Gene Therapy 8:1508-1513, 2001. Additionally, a vectors involving a DNA/ligand/polycationic adjunct coupled to an adenovirus is described in Curiel, D. T., Nat. Immun. 13:141-164,1994.

It will be appreciated by one skilled in the art that other viral and non-viral vectors and methods of using viral and non-viral vectors are known and can be used in accordance with the present invention. These other vectors and methods are described in gene therapy treatises, such as Vector Targeting for Therapeutic Gene Delivery, Wiley-Liss, Inc. Hoboken, N.J. 2002, and Viral Vectors for Gene Therapy, Method and Protocols, Humana Press, Totowa, N.J. 2003.

The nucleic acid to be delivered to the RPE cell to induce and/or facilitate expression and/or secretion of the therapeutic protein can comprise any gene that induces expression of a therapeutic protein in the RPE cells. Examples of genes that can be delivered include the gene for aromatic amino-acid decarboxylase (dopa decarboxylase) (Genbank HUMDDC, Accession No. M76180), as well as the gene for GDNF (Genbank HUMGDNF02; Accession No. L19063), brain-derived neurotrophic factor (BDNF), nerve growth factor (NGF) (EMBL HSNGF2; Accession No. X53655, and/or other members of the neurotrophin factor family including neurotrophin (NT)-3 (Genbank HUMBDNF; Accession No. M37762) and NT-4 (Genbank HUMPPNT4P; Accession No. M86528). Other genes which can be used to induce expression of a therapeutic protein from the RPE cells can be used in accordance with the present invention. These genes can be expressed individually or co-expressed with other genes so that more than one therapeutic protein can expressed.

The modified RPE cells in accordance with the present invention can be used for cell transplantation therapy and prevention of neurological disorders, such as Parkinson's disease, Huntington's disease, stroke, spinal cord injury, post-polio syndrome amyotrophic lateral sclerosis, epilepsy. It will be appreciated by one skilled in the art that other disorders and diseases including neural and non-neural disorders and diseases can be treated by the gene transfer agents of the present invention.

The modified RPE cells can be combined with pharmaceutical carriers, formulations and dosages to facilitate transplantation of the modified RPE cells to the target tissue. An assortment of delivery devices can be employed to deliver the modified RPE cells to the desired tissue in vivo. For example, the delivery device can include a hollow microneedle having an opening at its distal end through which the gene transfer agent can be injected. A delivery system can be coupled to control delivery of the gene transfer agent via the device.

When using the modified RPE cells to treat disorders and/or diseases, the therapeutic protein that is expressed or secreted from the modified RPE cells can be expressed within the target tissue for any suitable length of time, including transient expression and long-term expression. It is desirable that the therapeutic protein be expressed in therapeutic amounts for a suitable and defined length of time.

A therapeutic amount is an amount, which is capable of producing a medically desirable result in a treated animal or human. As is well known in the medical arts, dosage for any one animal or human depends on many factors, including the subject's size, body surface area, age, the particular composition to be administered, sex, time and route of administration, general health, and other drugs being administered concurrently. Specific dosages of proteins, nucleic acids, or small molecules can be determined readily determined by one skilled in the art using the experimental methods.

### EXAMPLES

The present invention is further illustrated by the following specific examples. The examples are provided for illustration and are not to be construed as limiting the scope or content of the invention in any way.

Dopaminergic Properties of Retinal Pigment Epithelial (RPE) Cells

Intrastriatal human RPE transplantation in parkinsonian animals and in PD patients have been immunologically tolerated, safe and efficacious in ameliorating parkinsonian symptoms. RPE cells are known to contain L-dopa and trace amounts of dopamine (DA) in vitro and have been shown to have in vivo 18F-dopa uptake in PET imaging studies. It is unclear whether L-dopa and DA production in RPE cells is catalyzed by tyrosine hydroxylase (TH) and aromatic amino acid decarboxylase (MDC) or through alternate enzymatic pathways. To investigate this mechanism, we tested human RPE cells grown in tissue culture for the presence of TH, tyrosinase (Tyr), MDC, dopamine transporter (DAT) and vesicular monoamine transporter (VMAT) by immunocytochemistry and western blot analysis. Rat Pheochromocytoma (PC12) cells were used as positive control for TH, MDC and DAT and as negative control for Tyr. SKNMC cells engineered to over express VMAT were used as positive control for VMAT and a human melanoma cell line was used as positive control for Tyr. Preliminary results suggest that RPE cells are negative for AADC when compared to PC12 cells, whereas they are positive for Tyr when compared with human melanoma cell line. RPE cells do not express DAT but do express VMAT. The western blot results repeatedly (3/3) confirmed the absence of AADC, and the presence of Tyr and VMAT in RPE cells. Immunocytochemical staining in PC12 cells revealed multiple patches of TH staining and uniform pattern of AADC staining, whereas staining in RPE cells were uniformly negative. Additional experiments are ongoing to confirm these results. A better understanding of the biosynthetic pathways involved in the production of L-dopa and DA in human RPE cells could provide an opportunity to enhance, regulate or otherwise modulate these cells.

### Introduction

Intrastriatal transplantation of RPE cells attached to microcarriers is a new cell transplantation technique that has been shown to effectively ameliorate parkinsonism in animal models of Parkinson's disease (PD) (Subramanian et al., Cell Transplantation, 2002; 7th INTR proceedings, Exp. Neurol. 2000) and in preliminary clinical trials in PD patients (Ray L. Watts, et. al., Neurology, 56 (Suppl. 3), P04.102. 2001, Parkinsonism & Related Disorders , 7 (Suppl.), S87, P-TU-305, 2001).

The mechanism/s through which RPE cells mediate behavioral recovery in parkinsonism is unclear. RPE cells are known to produce numerous growth factors such as platelet-derived growth factor, epidermal growth factor, and vascular endothelial growth factor. However, histological sections of transplanted brains show no evidence of host sprouting towards the graft site suggesting that the behavioral recovery is not mediated though growth factors.

Another possible mechanism of action of RPE transplants is through the secretion of dopamine (DA) or DA precursors into the parkinsonian striatum. DA production is mediated by tyrosine hydroxylase (TH) and dopa decarboxylase (DDC/AADC) in neurons. In most DA producing neurons, DA is sequestered within vesicles though the vesicular monoamine transporter (VMAT) and secreted DA is resorbed via the dopamine transporter (DAT). DA is also produced as an intermediate in the melanin production pathway. Here we present preliminary data on the dopaminergic properties of RPE cells grown in tissue culture. As controls, we used known dopaminergic cell lines like rat pheochromocytoma cells (PC-12), rat fetal ventral mesencephalon (FVM) primary tissue cultures and unilateral 6-OHDA lesioned rat brain sections through the striatum. In addition we used a neuroblastoma cell line engineered to overexpress VMAT-2 (SKNMC) as a positive control for VMAT.

### Materials and Methods

Cell Culture

Cells lines were purchased from ATCC (Manassas, Va.), specifically human retinal pigment epithelial (ARPE-19, #CRL-2302), human neuroblastoma (SK-N-MC, #HTB-10), human melanoma (#CRL-1424), and rat adrenal gland pheochromocytoma (PC-12, #CRL-172) cells. The cells were either grown in 6-well plates on glass cover slips (for immunofluorescence) or in culture flasks (for westerns) at 37° C.+5% CO2.

FVM tissue was harvested under aseptic conditions from E13 pregnant rats. FVM was aseptically microdissected out and pooled in F12: DMEM+10% FBS media until all dissections were complete. Using a 30-gauge needle, the tissue was drawn up and expelled to create smaller tissue “chunks” into individual petri dishes. The tissue was allowed to grow at 37° C.+5% CO2 for 3-7 days until fixation.

Immunofluorescence

The cells were grown to confluence according to ATCC recommendations, at which time they were fixed with 4% paraformaldehyde for 20 min at room temperature washed. FVM tissue was treated similarly. Cells/FVM were permeablized for 1 min in 0.1% PBSTX and incubated in 1% blocking serum for 1 hour in PBS. Primary antibody incubations were done in PBS containing 3% normal donkey serum and 0.05% Triton-X overnight on a shaker table at 4° C. with the following concentrations: tyrosine hydroxylase 1:1K (mouse anti-TH, PelFreeze), dopamine decarboxylase 1:500 (rabbit anti-DDC, Chemicon), vesicular monoamine transporter 1:1K (rabbit anti-VMAT2, Chemicon). At this time the cells/FVM were washed in PBS and incubated in the appropriate biotinylated secondary antibody (1:500, Jackson ImmunoResearch) for 1 hour in PBS. After PBS washes, cells were either incubated in Avidin-Texas Red or Avidin Fluorescein (1:500, unless otherwise noted, Vector Laboratories) for 1 hour for the fluorescent detection. Fluorescent cells were mounted with DAPI (Vector Laboratories).

Western Blot

Cells grown in flasks were harvested by trypsinization and sonicated in buffer containing 50 mM Tris HCL (pH 7.5), 200 mM NaCL, 5 mM EDTA, 40 mM Tris glycerophosphate, 0.2 mM sodium orthovanadate,1 mg/ml leupeptin, 1 mM pepstatin and 1 % Triton-X (chemicals from Fisher Scientific and Sigma). The lysates were centrifuged at 14,000 RPM for 15 minutes at 4° C. Total protein was estimated using BCA protein assay reagents (Pierce, Rockford, Ill.). 200 mg protein of each cell lysate was electrophoresed on a 10% SDS-polyacrylimide gel under reducing conditions. Prestained Kaleidoscope protein molecular weight standards (BioRad Laboratories) were included as markers in each gel. The separated proteins were blotted onto PVDF membranes (BioRad Laboratories) and were stained for the presence of TH (1:1K, PelFreeze), DDC (1:1K, Chemicon) and tyrosinase (1:1K, Chemicon). After a 24 hour incubation in the primary antibodies, the blots were incubated for 1 hour in a secondary appropriate to the primary IgG-HRP (1:5K, Jackson ImmunoResearch). The blots were developed using ECL+kit (Pharmacia) and exposed to x-ray film for 10 minutes-24 hours in the dark.

### Summary of Results

Immunohistochemistry

In comparison to PC-12, FVM and rat brain tissue RPE cells appear to have minimal TH staining. In comparison to PC-12, FVM and rat brain tissue RPE cells have no DDC staining (=background). In comparison to SKNMC cells engineered to overexpress VMAT-2, RPE cells appear to have significant staining for VMAT-2 RPE cells do not appear to have DAT Western blot analysis. In comparison to PC-12 cells RPE cells have mimimal TH protein. In comparison to melanoma and PC-12 cells, RPE cells had no detectable band for AADC. In comparison to melanoma and PC-12 cells RPE cells appear to have a well delineated band for tyrosinase. In comparison to SKNMC cells engineered to overexpress VMAT, RPE cells appear to have a well delineated band that corresponds to VMAT-2 (previously presented at INT-7, Subramanian et al., 1999).

### Conclusions

Our results support the notion that behavioral benefits following striatal ARPE transplantation is most likely mediated by dopa release locally into the host striatum. This dopa production is most likely a by product of melanin synthesis within the ARPE cells. Additional experiments to confirm this hypothesis are ongoing. We confirm that ARPE cells have VMAT2, which suggests that ARPE cells sequester dopa within vesicles. This is corroborated by our previous research observation that ARPE transplant locations have highly specific 18F-dopa uptake. Our results also indicate that the classic TH-mediated neuronal DA synthesis pathway does not appear to play a major role in DAergic metabolism in ARPE cells.

Recombinant Lenti-Viral Mediated Stable Transgene Expression in Retinal Pigment Epithelial (ARPE-19) Cells

We have successfully transplanted human retinal pigment epithelial (RPE) cells into animal models of Parkinson's disease (PD) and in PD patients to ameliorate symptoms with very little host immune response. RPE cells are known to produce a number of biologically active substances including L-dopa and neurotrophic factors. These properties make RPE cells a good candidate for ex-vivo gene therapy. We tested the feasibility of using a recombinant lentiviral (rLV) vector to introduce new genes into ARPE-19 cells (human RPE cells from a 19 year old donor, ATCC). A rLV (Lenti-HRZ-GFP-WPRE-Sin 8) vector was used to introduce green florescent protein (GFP) into ARPE-19 cells. Optimal functional titer was found to be 1.8×10⁸ TU for infecting 2.5×10⁵ ARPE cells. At 72 hours post infection, 27% of cells were found to be GFP positive. The intensity of GFP expression in ARPE cells was very high when compared recombinant MLV based retroviral and non viral gene transfer methods. GFP expression level remained stable up to 8 weeks with no evidence of morphological transformation or change in growth characteristics in the infected ARPE-19 cells. Additional tests for stable long term expression of GFP in vitro and in vivo in these ARPE-19 cells are ongoing. Our results suggest that rLV vector mediated ex-vivo gene therapy is an efficient and safe method for modifying ARPE-19 cells and that such methods could be used to modulate production and/or secretion of biologically active substances from these cells.

### Introduction

Transplantation of dopaminergic (DArgic) cells into the denervated striatum is an experimental approach to reduce symptoms associated with Parkinson's disease (PD). Previous studies from our laboratory have shown that RPE cells attached to microcarriers can be transplanted safely into PD patients without any systemic immunosuppression and that such transplants appear to ameliorate PD symptoms without any short-term risk of disabling side effects. RPE cells transplanted into the striatum appear to take up ¹⁸F-dopa (Subramanian T., et al., INT7 proceedings, 1999) in PET imaging studies. Taken together these findings suggest that RPE cell transplants have some dopaminergic properties (Venkiteswaran K, et al., INT-8, 2002). RPEC have been reported to contain tyrosinase (Dryja T P, OOVJ, 1978) and tyrosine hydroxylase (TH)-like activity (Smith S B, EER, 1998). RPE cells appear to lack the enzyme L-aromatic amino acid decarboxylase (AADC/DDC), which is the catalyst for the conversion of dopa to dopamine.

Therefore, we tested the feasibility, safety and efficacy of introducing the MDC and green florescent protein (GFP) genes into RPE cells (RPE-AADC and RPE-GFP). We tested non-viral, recombinant MLV-based retroviral and lentiviral protocols and compared them.

### Materials and Methods

Cell Culture

Retinal pigment epithelial cells (ARPE-19) were obtained from ATCC. Rat fetal ventral mesencephalon were obtained from E-13 time-pregnant rats through aseptic dissection. Adult brain tissue sections were obtained from unilateral 6-OHDA lesioned rats. Human embryonic kidney cells (29310A1, Immgenex, CA) were grown according to the manual. ARPE-19 cells in log phase growth state were used to transfect using non-viral method or using retroviral methods. For lentiviral infection, ARPE cells maintained in confluent state for at least 12 weeks were used.

Non Viral Transfection

pCDNA3.1 zeo+plasmid was used as the non-viral vector to clone GFP gene. The transfection was done according to Boeckman et al (Promega neural notes 1996,11(2) 13-15). The GFP gene was cloned at the Bam HI and Xho I sites on pCDNA3.1 zeo^{+.} RPE cells were transfected using Transfast™ Transfection Reagent (TF×50, Promega Inc.). Briefly, the plasmid containing GFP gene alone were mixed with TF×50, vortexed and placed at room temperature for 15-45 minutes to allow the lipid and DNA to associate. ARPE-19 cells in log phase growth were incubated with the lipid/DNA mixtures at 37° C. for 2-24 hours. The gene expression was assayed 2, 7, 9, 14 and 28 days after transfection by visualizing under an indirect florescent microscope and green cells were visually counted under a grid on each day.

Retroviral Transfection

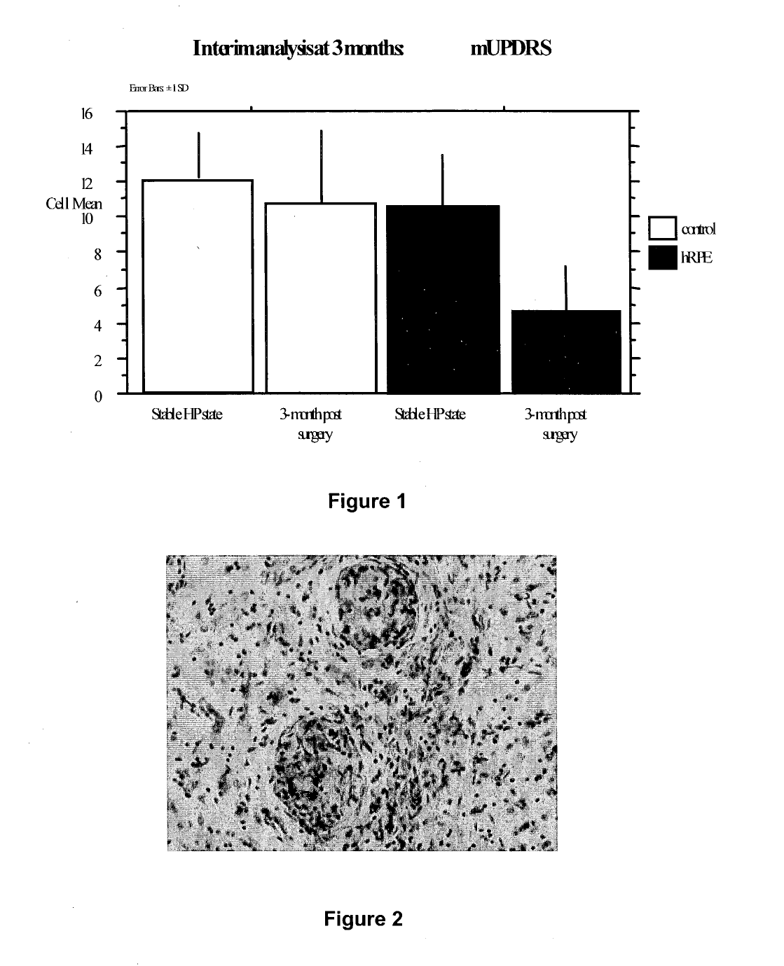

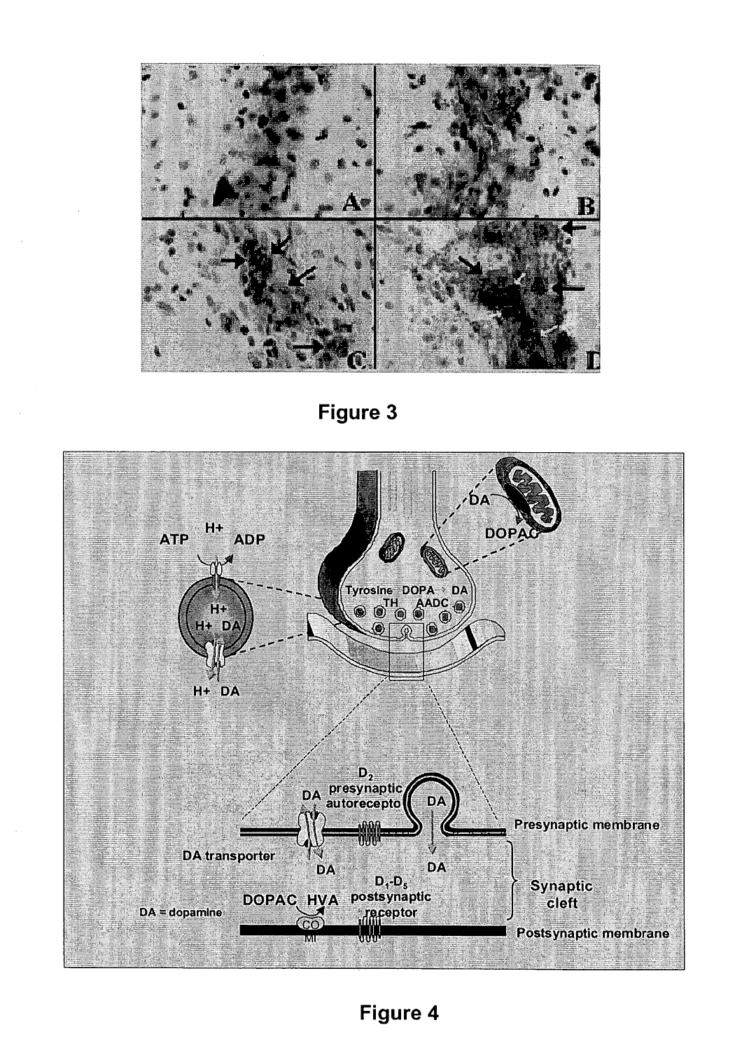

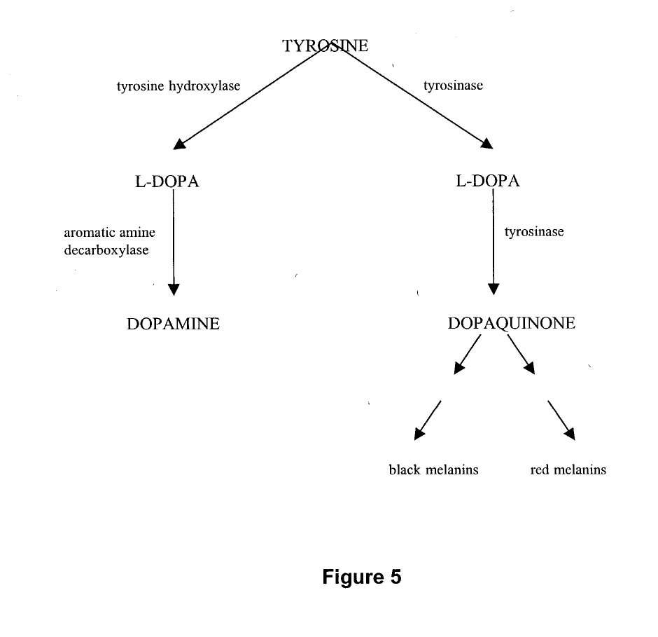

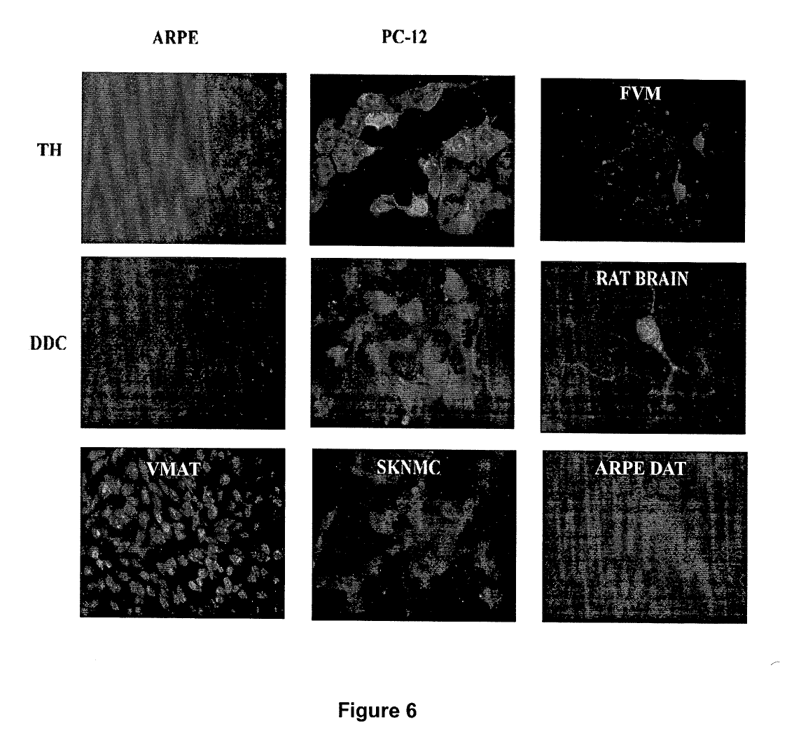

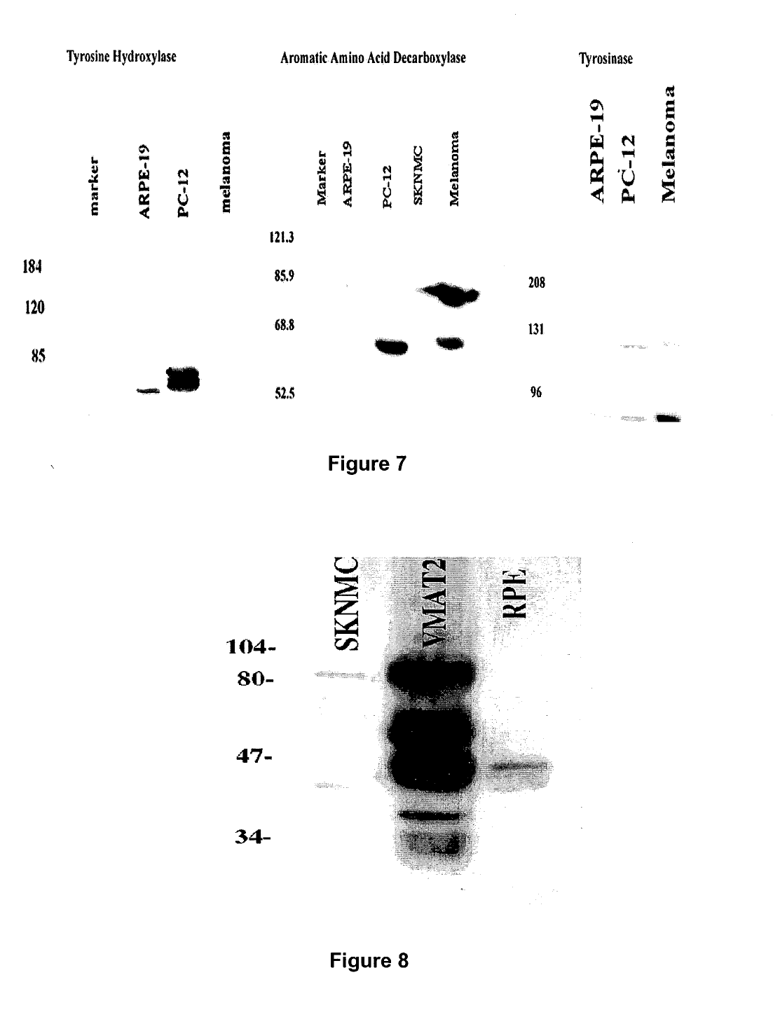

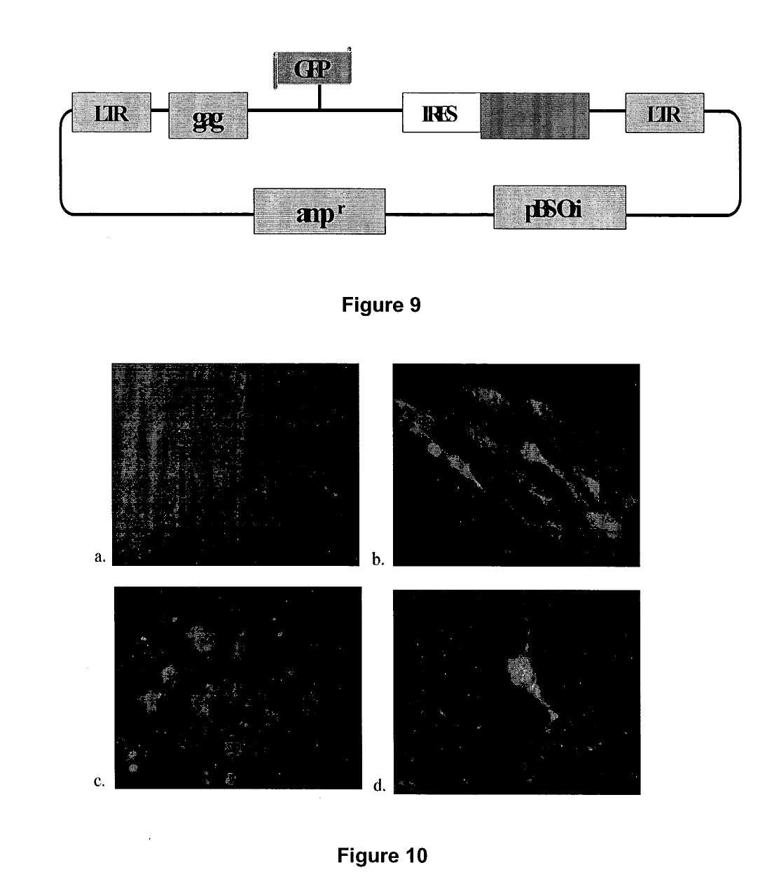

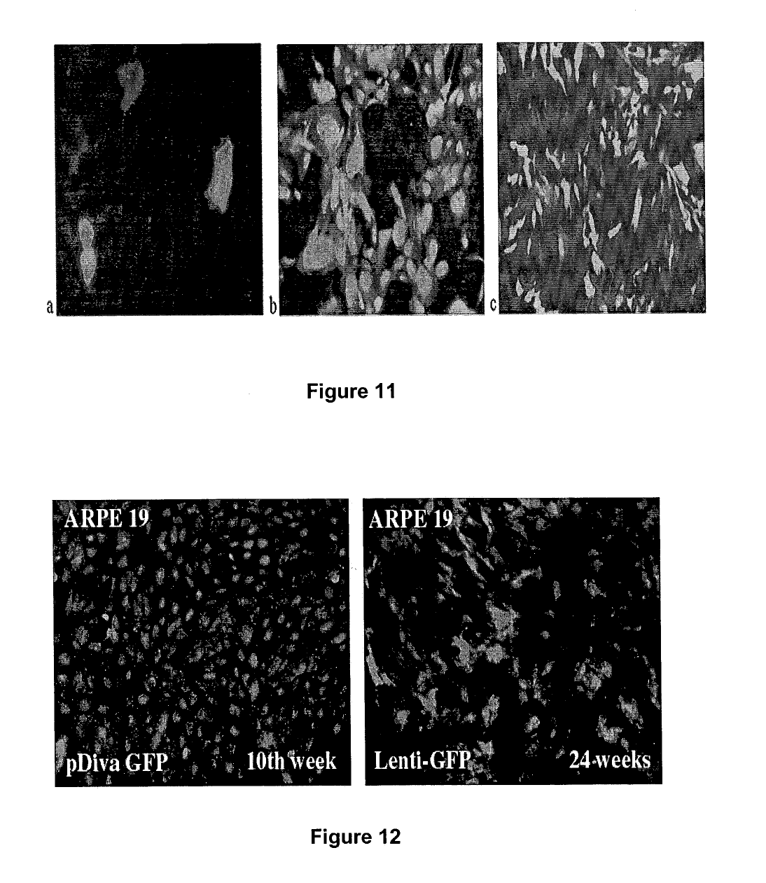

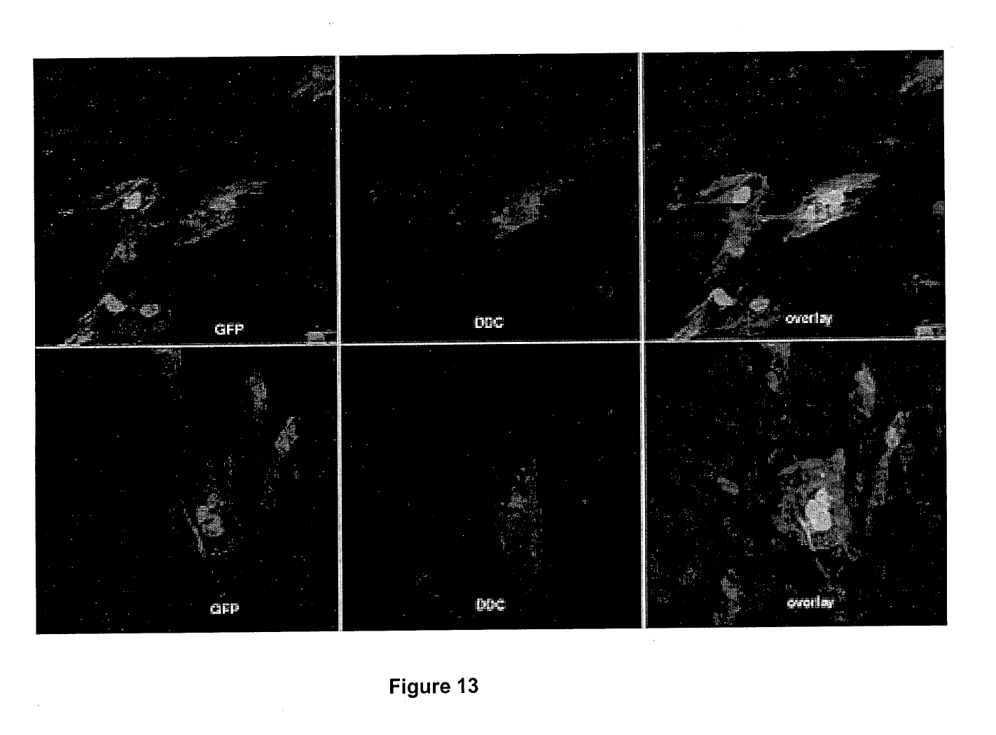

We used pDiva, a replication deficient MLV based retroviral vector generating plasmid in these experiments (gift from Dr. Kevin Pumiglia, Albany Medical College, N.Y.). The GFP gene was introduced into Bam Hi and XhoI sites of the vector pDiva, in which the puromycin resistance marker is driven by an internal ribosome entry site (IRES). See FIG. 1. 29310A1 cells were transfected with pDiva GFP using the calcium phosphate protocol [Naviaux et al 1996]. Recombinant retroviral particles were collected after 48 hours. This supernatant containing viral particles was used to infect ARPE-19 cells at 32° C. in presence of polybrene (PNAS; 90(18):8392-6). ARPE-19 cells transfected with retrovirus were transferred to selection medium containing puromycin after 72 hours. This procedure allows selection of transfected cells. Thereafter, all transfected cells were allowed to grow to confluence. Cultures were visualized for GFP under a florescent microscope and counted periodically for 6-8 weeks.

Lentiviral Protocol

Plasmid construction: All cDNAs will be introduced into the transfer lentiviral vector pHR'CMV-GFP (green fluorescent protein; provided by D. Trono) that has been modified to contain the woodchuck hepatitis virus posttranscriptional enhancing element (WPRE, gift of T. Hope) and a polypurine tract (ppt) cloned upstream of the CMV-GFP cassette. The human AADC cDNA was subcloned in the place of GFP from an existing AAV expression vector PMDAADC.

Virus Production

Lentivirus was produced as described by Naldini et al using the helper plasmid pCMVR8.91 and the Vesicular Stomatotitis Virus glycoprotein (VSV-G) pseudotyping envelope expression plasmid PMDG. Briefly, pCMVR8.91 and PMDG were cotransfected along with the transfer vectors pHR'CMVhAADC and PHR'CMVGFP into 293T cells using standard calcium phosphate co-precipitation methods. Transfections proceeded for 5 hours after which media was replaced. Media containing virus was harvested at 60 and 130 hours after transfection. Media was filtered to remove cell debris and virus was concentrated by centrifugation and resuspension in 1/50^{th} volume PBS. Functional titers were determined by infection of serial diluted virus onto 293T cells followed by immunocytochemistry for expressed cDNAs or flow cytometry measuring GFP-fluorescing cells. Cell counts of immunopositive or GFP-fluorescent cells were used to determine approximate titer.

Infection of ARPE Cells

ARPE-19 cells were grown to confluence and allowed to “mature” in the confluent state for 8 weeks. These cells had formed a single layer of cells with characteristic cobble stone appearance, contact inhibition and melaninization. Initially cells were infected with lenti-GFP overnight (˜18 hours). Thereafter, medium was changed and the cells visualized periodically for the presence of green florescence. One half of the RPE cells infected with lenti-GFP were then infected with lenti-AADC for 24 hours. After medium change, cells were fed every week with fresh medium and periodically visually examined.

AADC/DDC Immunocytochemistry

ARPE cells infected with lenti-GFP and lenti-DDC were allowed to grow to confluence and fixed using 4% paraformaldehyde. Immunostaining was done using anti-dopa decarboxylase polyclonal (Chemicon, 1:500) overnight at room temperature as previously described (Leff S E, et. al., Neuroscience. 1999;92(1):185-96). Briefly, cells were incubated overnight in the primary antibody, washed and exposed to biotinylated anti-rabbit IgG (1:200) followed by incubation with avidin-rhodamine (1:500). The cells were visualized in a fluorescence microscope and/or confocal microscope.

### Results

Non Viral Method

A very small percentage (<1%) of ARPE cells were found to have green florescence. The green florescence was noted in these cells on day 4 and thereafter this remained stable for several weeks.

MLV Based Retroviral Infection

ARPE-19 cells plated at 50,000 cells/well in 6 well plates and infected with MLV based retroviral vector (pDIVA-GFP) overnight (˜18 hours). On day 5, the presence of GFP was detectable in approximately 10% of the cells. Puromycin selection was done on day 5 that resulted in 99% of the surviving cells to be GFP positive on day 10. Stable expression was seen up until 10 weeks.

Lentiviral Infection

We infected ARPE-19 cells grown to confluence for 4 weeks with lenti-GFP overnight, washed and then exposed the cells to lenti DDC overnight again, washed and fed fresh medium. On the 5^{th} day approximately 10% of the cells were GFP positive and these cells were fixed and stained for DDC. Approximately, 15-20% of the GFP positive cells were DDC positive. To further clarify the optimal virus titer, we checked serial dilutions of lenti-GFP into RPE cells. The highest concentration (1×10⁸ TU per well in a 6 well plate) gave the highest GFP positivity (˜10%). For lenti-DDC the highest positivity was seen in the highest concentration of viral particles (1×10⁸ TU per well in a 6 well plate). In parallel experiments stable GFP expression was noted up to 24 weeks.

TABLE 1
Immunofluorescence in ARPE-19 cells
infected with lenti-GFP followed by lenti-DDC

| | | | Percentage of DDC |
|---|---|---|---|
| Virus used in | Percentage | Percentage | among GFP positive |
| TU | GFP | DDC | cells |
| 1 × 10⁸ | 10.01 | 4.22 | 15.37 |
| 1 × 10⁴ | 4.46 | 2.32 | 45.24 |
| 1 × 10² | 2.66 | 1.02 | 17.73 |
| 10 | 0.91 | 0.42 | 21.08 |
| 5 | 0.56 | 0.29 | 20.57 |
| 3 | 0.2 | 0.06 | 10.42 |

TABLE 2
Comparison of different methods of
transfection in ARPE cells for GFP

| | Percentage expression of GFP | | |
|---|---|---|---|
| | after | | |
| Method of Transfection | 24 hours | 10 weeks | 24 weeks |
| Non-viral | 0.001 | N/a | N/a |
| Retroviral (mMLV | 10 | 100 | 100 |
| based) | | (selected) | (selected) |
| Lentiviral | 10 | 27 | 27 |

### Conclusions

ARPE cells can be safely transfected with GFP using non-viral, MLV based retroviral and lentiviral methods. There were no obvious deleterious effects to the cell cultures from the 3 methods of transfection. Non-viral method appeared to be the least efficacious. MLV based recombinant retrovirus (pDIVA-GFP) after puromycin selection allowed nearly 100% of the cells to become GFP positive. However, “shut off phenomenon” may be a limiting factor in the utility of this method for cell transplantation. Seqential double transfection with 2 separate lentiviral vectors for GFP and DDC was successful in introducing both genes into a small percentage of ARPE cells. Improved recombinant lenti viral vectors with bidirectional promoters that control GFP and DDC may be more efficacious in introducing these 2 genes into RPE cells. Behavioral effects of transplanting genetically engineered ARPE cells are ongoing. Moreover, the modified RPE cells as described can be modified to form spontaneous microspheres, which can be visualized under microscope as well as after transplantation into animal. The microspheres comprise a non-cellular inner core and a cellular outer core.

From the above description of the invention, those skilled in the art will perceive improvements, changes and modifications. Such improvements, changes and modifications within the skill of the art are intended to be covered by the appended claims.

1. A transplantable composition comprising retinal pigment epithelium cells modified to induce expression and/or secretion of a therapeutic molecule, that can be used to prevent and/or treat disease.

2. A method of prevention and/or treatment of a neurological disorder, the method comprising the step of transplanting into tissue to be treated retinal pigment epithelium cells modified to induce expression and/or secretion of a therapeutic molecule that can be used to treat the neurological disorder
